(19) United States
(12) Patent Application Publication
(10) Pub. No.: US 2009/0073386 A1
(43) Pub. Date: Mar. 19, 2009

(54) ENHANCED HEAD MOUNTED DISPLAY

(76) Inventors: G. Timothy Petito, Safety Harbor, FL (US); Howard B. Purcell, Jacksonville, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/209,309

(22) Filed: Sep. 12, 2008

Related U.S. Application Data
(60) Provisional application No. 60/972,565, filed on Sep. 14, 2007.

(51) Int. Cl. A61B 3/032; G02B 27/01

(52) U.S. Cl. 351/243; 345/8

(57) ABSTRACT

This invention discloses methods and apparatus for generating an ophthalmic lens with at least a portion of one surface free formed from a reaction monomer mix.

This application is a non-provisional filing of a provisional application, U.S. Ser. No. 60/972,565, filed on Sep. 14, 2007 and claims priority to the Provisional Application U.S. Ser. No. 60/972,565, filed on Sep. 14, 2007.

### FIELD OF USE

The present invention relates to an image display apparatus that presents a virtual image to an observer, and also relates to a head-mounted display with enhanced resolution.

### BACKGROUND

Vision is the major component of information gathering for human beings in many scenarios. However, our assessment of vision has remained relatively static for more than one hundred years and centers primarily on the ability to see “20/20”, as originally introduced by Dr. Snellen in the 1860's.

The modern world additionally introduces environmental stresses to bear on the human experience that may not be adequately addressed by a simple 20/20 assessment. For example, an increase in the speed of objects around us and our own travel, as well as the need to focus on small objects or text in varying degrees of contrast and glare create new challenges to the assessment of satisfactory sight. In essence, in order to rapidly and accurately gather useful information, human eyes must be oriented in a way that brings needed visual detectors in proximity with the field where the needed information resides, and do so in a timely fashion.

Suitable assessment of what is satisfactory eyesight is difficult with traditional apparatus, such as the Snellen Test mechanism. Even if such equipment could be made to provide testing protocols relevant to the modern experience, the cost of such equipment is prohibitive to much of the world's population. A full compliment of equipment in a typical office of a modern day optometrist or ophthalmologist simply cannot be afforded by third world economic systems.

In addition, the use of virtual space in eye care is currently unknown. This may be due, in part, to the perception by the industry that such technology would be prohibitively expensive. Prior to the present in invention, visual systems with a resolution necessary to effectively assess vision at an accuracy better than about 20/40 would be prohibitively expensive. In addition, even if such equipment were to be available, it has not been adapted to the realm of diagnosis or treatment.

### SUMMARY

Accordingly, the present invention includes methods and apparatus for providing relatively low cost assessment of human sight in a manner that reflects real world stresses experienced by a patient.

The present invention provides a head mounted display with optical characteristics suitable for assessing a patient's sight in a manner consistent with the patient's actual visual challenges.

### DESCRIPTION OF THE DRAWINGS

FIG. 1A illustrates a single high resolution image superimposed over another image.

FIG. 1B illustrates double high resolution images superimposed over another image.

FIG. 2 illustrates some embodiments for forming a superimposed high resolution image portion.

FIG. 3 illustrates some embodiments with additional optics.

FIG. 4 illustrates a controller connected to a head mount display unit.

FIG. 5 illustrates a controller that may be used in some embodiments of the present invention.

### DETAILED DESCRIPTION OF THE INVENTION

According to the present invention, a head mounted display (“HMD”) is provided with adequate optical resolution and eye tracking apparatus to provide a platform for dynamic testing parameters of the visual system. Some tests may correspond, for example, with traditional clinical testing and additional tests may include tests heretofore unavailable on a widespread basis.

Additional tests recognize vision as a significant component of information gathering in environments where a patient requires speed. The present invention provides methods and apparatus for placing visual detectors in proximity with the field where the needed information resides and allows the patient's eyes to be oriented in a way that emulates actual life experiences. Enhanced tests can include, for example, foveal fixation of a stable object.

The present invention provides a HMD with sufficient resolution and programmed displays to assess high spatial frequency information, such as detail, or acuity in a monocular mode and also one or more of: color; depth (i.e. vergence mediated or stereopsis (Z axis) both of which utilize binocularity); contrast; contour; spatial localization (X-Y); and stability. One or more of the preceding may be assessed synchronously or simultaneously.

Relatively high resolution is optimal for at least some of the tests administered via the HMD. According to some embodiments, a HMD display provides both standard resolution and enhanced resolution portions. A HMD can utilize a first image source for a comprehensive display at standard resolution and a second image source for a second image display at enhanced resolution. The first image display and the second image display are superimposed over each other to provide at lest a portion of an aggregate display in relatively high resolution. Some embodiments can include an organic light emitting diode (“OLED”) system as one or both of the first image source and the second image source.

In addition, to testing according to the present invention, the HMD can be used for training in a virtual space. The training can be static in order to follow a set regimen; or dynamic, whereby a subsequent training level or exercise is based upon recorded performance of a preceding performance.

The HMD itself can be controlled by a computing device. Executable software on the computing device can be used for one or more of: producing tests; produce test parameters; deliver instructions to a patient describing test regimens; control test parameters in an HMD; gather patient responses and produce reports.

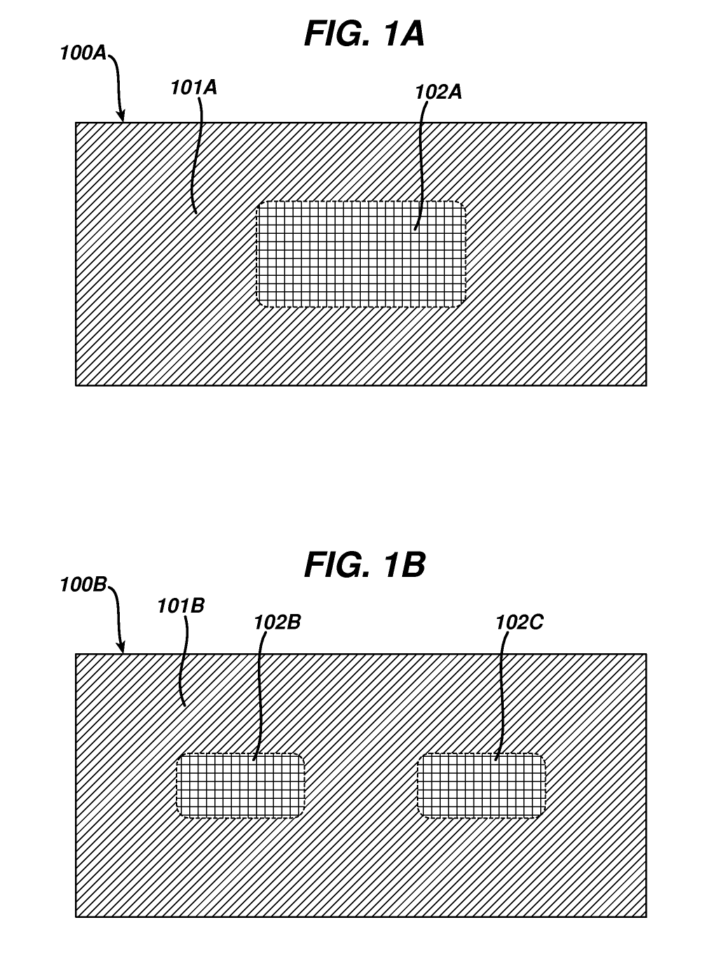

Referring now to FIG. 1, a HMD 100A can include two or more image portions 101A-102A. Each image portion may have a different resolution, with at least one image portion including sufficient resolution to assess high spatial frequency information and assess eye metrics. As illustrated, two image portions are shown, however, embodiments may also include three or more image portions. A first image portion 101A provides a relatively lower resolution over a broader display area. A second image portion 102A includes a relatively higher resolution over a smaller display area.

As stated above, additional higher resolution display areas 102B-102C are within the scope of the present invention, and may include, for example two high resolution areas 102B-102C with respective high resolution area 101B designated for each eye of a user wearing a HMD.

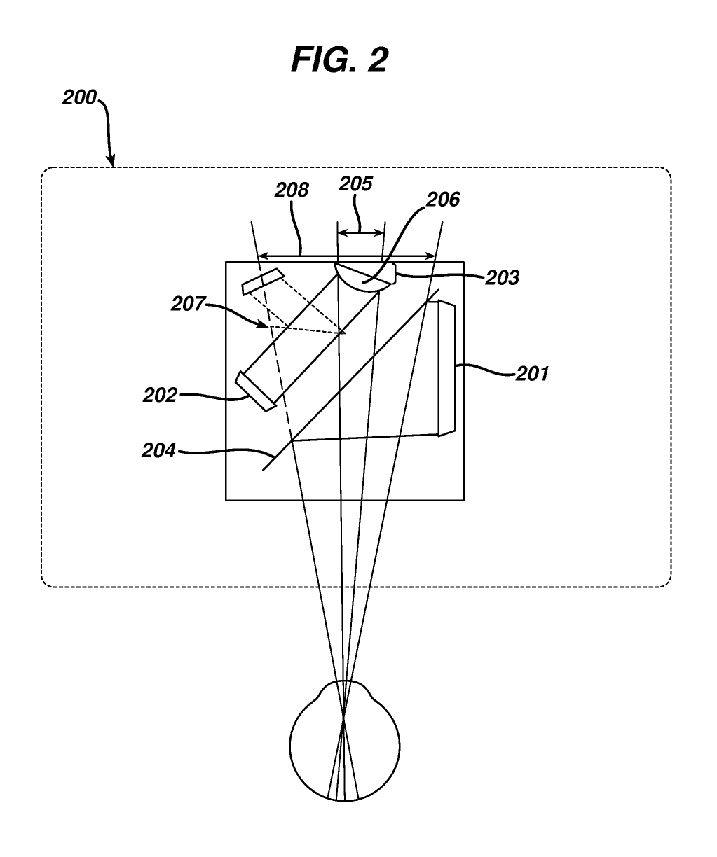

Referring now to FIG. 2, components of a HMD 200 according to some embodiments of the present invention is illustrated. The HMD 200 is constructed to scale to be worn by a human patient. The HMD includes a first image generation portion 201, such as for example an OLED panel. Other image generation apparatus may also be utilized, such as, for example other light emitting diode designs. The first image generation apparatus 201 generates an image displayed on a first image display portion 101A-101B.

A second image generation apparatus 202 can also include also an OLED panel or other image generation device. An optical minimizing apparatus 206 is positioned to receive output from the second OLED panel 202 and increase the resolution of a display of output from the second OLED panel 202. The optical minimizing apparatus 206 can include, for example a convex mirror which is positioned to minify an image produced by the secondary OLED display 202 and thereby increase a resolution of the image produced by the secondary OLED display 202. The pixel size of the minified image that comprises the second image portion 102B can thereby be a function of the original pixel size of the secondary OLED display 202; the curvature of the mirror and the distance of the mirror from the OLED display 202. One specific example of a commercially available OLED display 201-202 can include the W05 display unit available from eMagin Corp.

Some embodiments can include, for example a convex mirror that is used to minify the image of the secondary OLED display 202 and thereby increase resolution via a minification factor of 6. A minification factor of 6 provides a resolution of 0.4 arcmin per pixel, beginning with a 2.4 arcmin per pixel size for the native secondary OLED display 202.

Additional embodiments can include the use of a mirror 206 with adaptive optics. The adaptive optics are operative to change the curvature of the mirror, which in turn results in a change in the minification factor of an image reflected from the mirror.

Still further embodiments can include a mechanism for providing a variable distance between the secondary OLED display 202 and the convex mirror 206. The variable distance may be used in concert with, or in place of an adaptive optics mirror to vary the minification factor of an image reflected from the mirror.

A beam splitter 204 can be used to overlay an image from the first OLED system 201 and the minified image from the second OLED system 202. The overlaid images can be presented to a user wearing an HMD containing the first OLED display 201 and second OLED display 202.

In some embodiments, the beam splitter 204 may also be used to attenuate the luminance from one or both of the first OLED display 201 and the second OLED display 202. In some embodiments, attenuation of each image can be a predetermined amount, such as, for example, a 50% attenuation of a first image and 50% attenuation of a second image. Other embodiments can include disparate attenuation of a first image and a second image, such as, for example 60% of a first image and 40% of a second image. In still other embodiments, in an active beam splitter, such as for example, an active LED beam splitter, the percentages of attenuation of transmitted light from the first or second image may be varied as needed. Some preferred embodiments therefore include attenuation associated with the first OLED display 201 and the second OLED image 202 that is controllable via software or via a user activated control.

The image of the first OLED display 201 will display in a relatively larger field of view (“FOV”), in some embodiments, the FOV can be approximately 40 degrees. Generally available OLED displays can support a resolution of approximately 2.4 arcminute per pixel 208. The second OLED display 202 will present a smaller FOV, such as, for example 6.5 degree diagonal FOV. The second OLED image 202 will also provide a higher resolution, such as, for example a resolution of 0.4 arcminute per pixel 205.

In some embodiments, each eye of a user will have a clear line of sight to the smaller, higher solution field generated by the second OLED image 202. Generally the first OLED image 201 provides a visually immersive environment and the second OLED image 202 provides high resolution areas and optotypes useful for high level visual testing.

In another aspect, a servo control motor 203 207 may be operational to tilt one or both of the convex mirror 206 and a flat mirror. The change in position will deflect the optical path of the high resolution field in relation to the low resolution background.

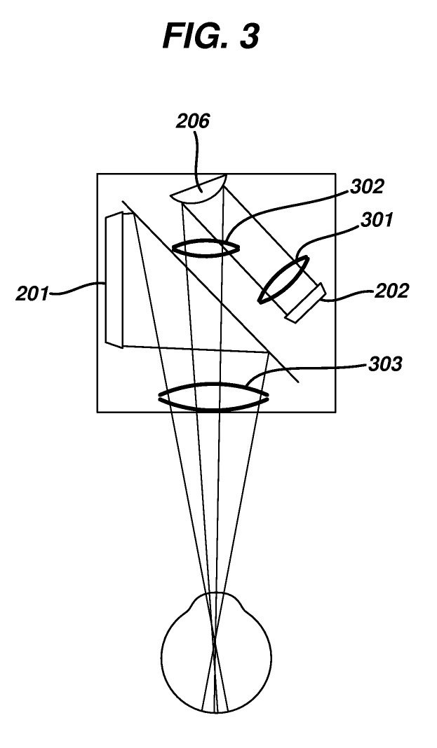

Referring now to FIG. 3, in still another aspect, in some embodiments, additional optics may be utilized for one or more of: correcting for differences in optical vergence between the first OLED display 201 and the second OLED display 202; correcting for ametropia of a user; and creating an optical stimulus to accommodation. Such additional optics can be placed, by way of non-limiting example, in one or more of: optics 301 in an optical path between the second OLED display 202 and the convex mirror 206; optics 302 between a convex mirror 206 and a beam splitter 201; and optics 303 after the beam splitter.

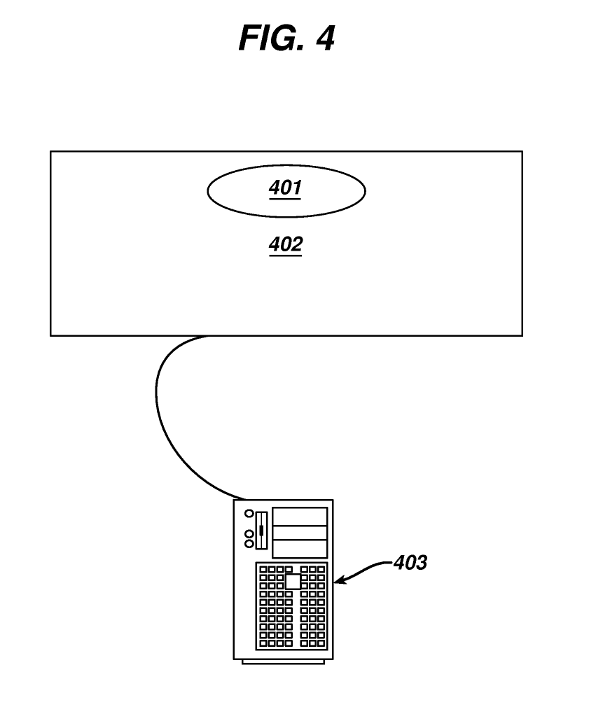

Referring now to FIG. 4, in some embodiments, an eye tracking apparatus 401 may also be incorporated into a HMD unit 402 with a visual system such as those described above. Eye tracking systems 401 are commercially available and provide for automated tracking of a line of sight of an eye. Eye movement tracking can be useful to provide for monitoring the response characteristics of the visually related motor components.

In some embodiments, a HMD 402 and computer device 403 providing controlled displays within the HMD 402 are operative to train visual performance in the virtual space by modeling specific visual scenes, and controlling the parameters and information which must be gathered from analyzing those visual scenes. Basic visual skills such as saccadic accuracy, pursuit speed, anticipation, vergence range, hand-eye coordination, stereoscopic sensitivity, suppression, etc. can be modified by training those skills. Perceptual and cognitive aspects of visual behavior can also be enhanced through practice within the virtual scenarios. Therefore, the benefits performance improvements usually ascribed to “practice” can also be achieved with the use of this device.

Some exemplary tests which may be implemented utilizing a system as described herein, can include, for example, the following:

VA: Visual Acuity: the specific optotypes can be anything that conforms to standards of 5:1 image size to detail size ratio, which could be “Landolt C” (standard optotype) or letter based as in “Snellen” acuity, or a hybrid as in “Broken Wheel” testing. Not only size, but contrast as in ETDRS, Baily-Lovey, or Peli-Robson tests, color, presentation duration, location, and movement of the optotypes (dynamic acuity) can be manipulated.

Dynamic Acuity (acuity on a dynamic target): The testing of acuity under dynamic conditions has meant different things to different groups up to now. This system will allow for testing of dynamic acuity in a variety of ways, which will lead to a standard, method, once comparisons can be made between competing options in this testing venue. Parameters which can be manipulated would include, speed, location, direction, target design, optotype design, optotype size, color, contrast, presentation duration, and any combination of these individual parameters.

CSF: Contrast Sensitivity Function—this involves testing the limits of detection of the individual for stimuli presented as gratings (sine-wave, square wave, gaussean, cosine squared, etc), letters, circular “bull's eye” targets, or any other luminance distribution pattern needed. The factors that can be manipulated are, luminance, contrast distribution, presentation duration, color, stationary vs. flickering or contrast reversal, spatial characteristics of the grating (i.e. size of the light and dark components of the target), location, and movement of the target.

Color Vision: Matching reference colors to a test stimulus to determine whether the individual has appropriate sensitivity to wavelength of light.

Cover Test: Presenting stimuli to each eye in the same position to evaluate whether the yes are directed in the proper orientation when the target is shown to the fellow eye only. It measures the presence of strabismus, or heterophoria and is a measure of the amount of vergence correction required for single binocular vision.

NPC: Near Point of Convergence: Measures the closest point that a person can binocularly fixate an object.

Stereopsis Distance: Random dot patterns would be utilized (which is the standard for near, but could also be in the format of a Howard-Dolman task, if desired. The parameters that can be manipulated include disparity, color, luminance, size, location, movement, stimulus duration, target configuration (i.e. picture used to present the disparity).

Stereopsis Near: Random dot tests as is the standard for current clinical tests, and with the same control on target parameters as listed for distance testing.

Stereopsis can be tested with vergence loads, either at distance or near. This will allow for tests of stereopsis while challenging the vergence system at increasing or decreasing loads by ramp changes, step changes, or hybrid changes of vergence. The IR eye tracking mechanism will monitor eye position.

Visual field: The extent of the world that can be seen by an eye without an eye movement.

Vergences: Eye movements which change the orientation the visual axes of the two eyes in opposite directions. (One eye to the right, the other to the left, or one eye up, the other down, etc).

Verssions: Eye movements which changes the orientation the visual axes of the two eyes in the same direction. Including one or both eyes to the right, or both left, or up, down).

Fixation Disparity (Horizontal & Vertical, DV/NV).

Fusional Status (1^{st}, 2^{nd} degree (worth dot) or amblyoscope targets).

Hess Lancaster.

Aniseikonia measurements.

Cyclotorsional measurements.

Reaction time.

Gaze Behavior & eye movement dynamics (free space and HMD).

Hand/Eye coordination.

Perceptual tests, such as, for example visual memory, figure ground, and discrimination.

Some embodiments of the present invention are capable of providing training visual skills and functions in a visually immersive artificial environment with control of environmental parameters.

In some embodiments, additional body movements may also be monitored and tracked. By way of non-limiting example, body movement tracking may include one or more of: head tracking, hand, foot, arm body, and other locations on the body of the patient, or objects they interact with can be and in certain applications would be monitored and utilized in the control and presentation of the virtual environment. This present invention will allow for complete control of all environment al factors which could influence the performance of the individual as related to the visual system, integration of the sensory systems with each other, and with the motor control systems, and motor response systems they employ in processing visual input, analyzing visual scenes, planning moor responses to visual stimuli and environments, and executing motor plans, including the monitoring, modification of motor planning and feedback loops involved in final response characteristics. Therefore both closed and open loop conditions will be possible, and under the control of the operator of the system.

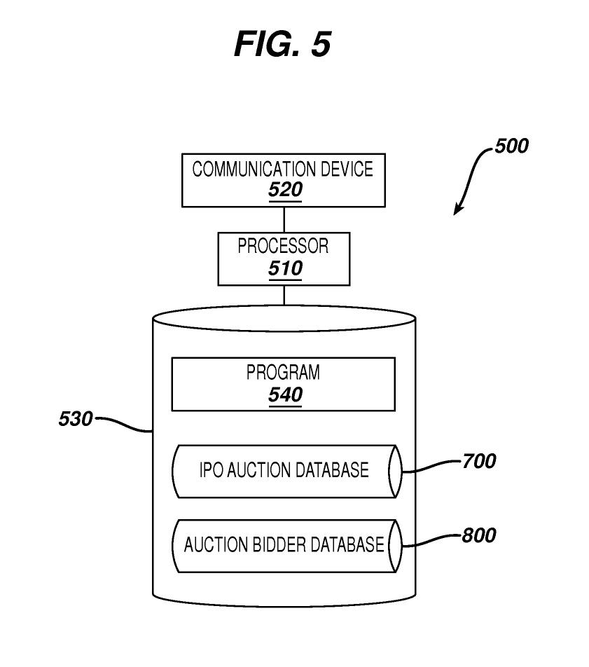

Referring now to FIG. 5, FIG. 5 illustrates a controller 500 that may be used to implement some aspects of the present invention. A processor unit 510, which may include one or more processors, coupled to a communication device 520 configured to communicate via a communication network. The processor 510 is also in communication with a storage device 530. The storage device 530 may comprise any appropriate information storage device, including combinations of magnetic storage devices (e.g. magnetic tape and hard disk drives), optical storage devices, and/or semiconductor memory devices such as Random Access Memory (RAM) devices and Read Only Memory (ROM) devices.

The storage device 530 can store executable software programs 515 for controlling the processor 510. The processor 510 performs instructions of the program 515, and thereby operates in accordance with the present invention. The storage device 530 can store related data in a database.

### CONCLUSION

The present invention, as described above and as further defined by the claims below, provides methods of processing ophthalmic lenses and apparatus for implementing such methods, as well as ophthalmic lenses formed thereby.

1. A head mounted display apparatus for conducting visual testing, the apparatus comprising:
a first light emitting diode display unit secure to a head mount and providing a first human readable display image;
a second light emitting diode display unit additionally secured to the head mount and providing a second human readable display image;
a beam splitter unit mounted in the head mount in a position capable of receiving two or more display images and displaying the two or more display images in a human recognizable form; and
a optical minimizer capable of receiving the second human readable display image and minifying said second human readable display image and redirecting said second human readable display image display to the beam splitter.

2. The apparatus of claim 1 wherein at least one of the first light emitting diode display unit second light emitting diode display unit comprises an organic light emitting diode.

3. The apparatus of claim 1 additionally comprising a processor for controlling the first light emitting diode display unit and the second light emitting diode display unit.

4. The apparatus of claim 1 wherein the optical minimizer comprises a convex mirror.

5. The apparatus of claim 4 wherein the convex mirror increases the resolution of the image from the second human readable display unit by a minification factor of 6 or more.

6. The apparatus of claim 4 wherein the convex mirror increases the resolution of the image from the second human readable display unit to provide a resolution of about 0.4 arcmin per pixel or higher resolution.

7. The apparatus of claim 6 wherein the second human readable display unit generates a display image at a resolution of about 2.0 to 2.8 arcmin per pixel.

8. The apparatus of claim 6 wherein the beam splitter is functional to superimpose the image from the second human readable display unit with increased resolution with the image from the first human readable display unit.

9. The apparatus of claim 6, wherein the image from the second human readable display unit with increased resolution is superimposed in a single area generally central to the image from the first human readable display unit.

10. The apparatus of claim 6, wherein the image from the second human readable display unit with increased resolution is superimposed in two areas with each respective area generally associated with a field of view of an eye of a user wearing the head mount.

11. Apparatus for displaying a human recognizable image in a human head mount, the apparatus comprising:
a first digital display unit secured within the head mount;
a second digital display unit secured within the head mount;
a controller comprising a processor and a storage for digital data; and
executable software stored on the storage for digital data and executable upon demand, the software operative with the processor to:
cause the first digital display unit a generate a human viewable image on a beam splitter within the head mount;
cause the second digital display unit to generate a human viewable image into a path of an optical minimizer, wherein said optical minimizer increases the resolution of the human viewable image generated by the second digital display unit onto the beam splitter; and cause the human viewable image generated by the second digital display to be super imposed over the image generated by the first digital display.
